(19) United States
(12) Patent Application Publication
(10) Pub. No.: US 2012/0035691 A1
(43) Pub. Date: Feb. 9, 2012

(54) SYSTEM AND METHOD FOR SECURING A LEAD IN A VESSEL

(76) Inventors: Bruce A. Tockman, Scandia, MN (US); Juan Gabriel Hincapie Ordonez, Maple Grove, MN (US); Shantha Arcot-Krishnamurthy, Vadnais Heights, MN (US); Lili Liu, Maple Grove, MN (US)

(21) Appl. No.: 13/198,505

(22) Filed: Aug. 4, 2011

Related U.S. Application Data
(60) Provisional application No. 61/371,057, filed on Aug. 5, 2010.

(51) Int. Cl. A61N 1/05

(52) U.S. Cl. 607/116

(57) ABSTRACT

A two-part system for securing and stabilizing a lead at a location within a patient's internal jugular vein adjacent a region of the vagus nerve to be stimulated is described. The two-part system includes a lead and a stent-like fixation member that is provided separate from the lead. The stent-like fixation member is used to secure an electrode region of the lead at a location within the internal jugular vein adjacent the vagus nerve. The stent-like fixation member urges the electrode region of the lead against the vessel walls of the internal jugular vein such that at least one electrode is oriented in a direction towards the vagus nerve. In one example, the stent-like fixation member includes a channel sized to receive and retain a portion of the lead therein.

### CROSS-REFERENCE TO RELATED APPLICATIONS

This application claims the benefit under 35 U.S.C. §119(e) to U.S. Provisional Patent Application No. 61/371,057, filed on Aug. 5, 2010, entitled “System and Method for Securing a Lead in a Vessel,” which is incorporated herein by reference in its entirety.

### TECHNICAL FIELD

The present invention relates to a system and a method for securing a medical electrical lead within a vessel so that neurostimulation can be delivered to an adjacent nerve. More particularly, the present invention relates to a two-part system and corresponding method for securing a neurostimlation lead within a patient's internal jugular vein at a location adjacent a target region of the vagus nerve within the carotid sheath so that neurostimulation can be delivered to a target region of the vagus nerve.

### BACKGROUND

The use of nerve stimulation for treating and controlling a variety of medical, psychiatric, and neurological disorders has seen significant growth over the last several decades, including for treatment of heart conditions, epilepsy, obesity, and breathing disorders, among others. For example, modulation of the autonomic balance with neural stimulation has been shown to be possible and have positive clinical benefits, such as protecting the myocardium from further remodeling and predisposition to fatal arrhythmias following a myocardial infarction (MI).

### SUMMARY

Example 1 is a lead system for stimulating a region of a vagus nerve from a location within an internal jugular vein, the system comprising: a medical electrical lead comprising a lead body extending from a proximal end to a distal end, a conductor extending within the lead body from the proximal end in a direction towards the distal end, and an electrode region having a first length along the lead body comprising at least one electrode located on the lead body and operatively coupled to the conductor, the electrode adapted to transvasculary deliver an electrical stimulation pulse to the target region of the vagus nerve from a location within the internal jugular vein; and a stent-like fixation member independently deployable of the lead, the stent-like fixation member having a second length extending in a direction along a longitudinal axis of the stent-like fixation member that is greater than the first length of the electrode region of the lead, the stent-like fixation member adapted to transition from a collapsed configuration for delivery to a location within the internal jugular vein to an expanded configuration, wherein in the expanded configuration, the fixation member is adapted to contact and engage the electrode region of the medical electrical lead such that the fixation member urges the electrode region including the electrode into a vessel wall adjacent the target region of the vagus nerve.

In Example 2, the system according to Example 1, wherein the elongated, stent-like fixation member includes a channel extending from a first end to a second end of the fixation member, the channel sized to receive and engage a portion of the lead body therein.

In Example 3, the system according to Example 2, wherein the channel has an inner diameter corresponding to the outer diameter of the lead body received therein.

In Example 4, the system according to Examples 2 or 3, wherein the channel is adapted to expand to an increased inner diameter so as to receive and engage the lead body therein.

In Example 5, the system according to any one Examples 1-4, wherein the stent-like fixation member is sufficiently resilient such that it wraps around and engages an outer surface of the lead body forming a channel around the lead body.

In Example 6, the system according to any one of Examples 1-5, wherein the elongated, stent-like fixation member includes a channel sized to receive and engage the lead body therein extending from a proximal end to a distal end of the fixation member and a tether coupled to the distal end of the channel and extending away from the stent-like fixation member in a proximal direction and the lead body further includes a lumen extending from the proximal end to the distal end of the lead body, wherein the lead body lumen facilitates delivery of the lead over the tether to be received channel of the stent-like fixation member.

In Example 7, the system according to any one of Examples 1-6, wherein the stent-like fixation member further includes a tether coupled to and extending in a proximal direction away from the stent-like fixation member and the lead body further includes a guide feature coupled to an outer surface of lead body such that the lead body is adapted to track alongside the tether.

In Example 8, the system according to any one of Examples 1-7, wherein the stent like fixation member further includes a tether coupled to and extending in a proximal direction away from the stent-like fixation member, the tether including or being formed from one or more conductors extending within the tether from a proximal end to a distal end of the tether and one or more electrodes located on an outer surface of the stent-like fixation member and operatively coupled to the one or more conductors extending within the tether.

In Example 9, the system according to any one of Examples 1-8, wherein the stent-like fixation member is balloon expandable.

In Example 10, the system according to any one of Examples 1-9, wherein the stent-like fixation member self-expanding.

In Example 11, the system according to any one of Examples 1-10, wherein the stent-like fixation member includes an insulative polymer coating or sheath.

In Example 12, the system according to any one of Examples 1-11, wherein the electrode region of the lead includes a pre-formed spiral region defining a lumen.

In Example 13, the system according to any one of Examples 1-12, wherein the electrode region of the lead includes a two-dimensional shape.

In Example 14, the system according to any one of Examples 1-13, wherein the stent-like member further includes a tether coupled to and extending away from a proximal end of the stent-like member and wherein the lead body further includes a lumen extending from the proximal end to the distal end of the lead body, wherein the lead body lumen facilitates delivery of the lead over the tether.

In Example 15, the system according to any one of Examples 1-14, wherein the lead body further includes one or more projections adapted to engage an outer surface of the stent-like fixation member.

Example 16 is a lead system for stimulating a region of a vagus nerve from a location within an internal jugular vein, the system comprising: a medical electrical lead comprising a lead body extending from a proximal end to a distal end, a lumen extending within the lead body from the proximal end to the distal end, a conductor extending within the lead body from the proximal end in a direction towards the distal end, and an electrode region comprising at least one electrode located on the lead body and operatively coupled to the conductor, the electrode adapted to transvasculary deliver an electrical stimulation pulse to the target region of the vagus nerve from a location within the internal jugular vein; and a stent-like fixation member independently deployable of the lead, the stent-like fixation member comprising a concave channel extending from a proximal end to a distal end of the stent-like member and sized and shaped to receive the lead body therein, the stent-like fixation member adapted to transition from a collapsed configuration for delivery to a location within the internal jugular vein to an expanded configuration, wherein in the expanded configuration, the stent-like fixation member urges the electrode region including the at least one electrode into a vessel wall adjacent the target region of the vagus nerve.

In Example 17, the system according to Example 16, wherein the stent-like fixation has a length when expanded extending in a direction along a longitudinal axis of the stent-like fixation member that is greater than a length of the electrode region of the lead.

In Example 18, the system according to Example 16 or 17, wherein the stent-like fixation member further includes a tether coupled to the distal end of the stent-like fixation member and extending away from the stent-like member in a proximal direction, wherein the lead body lumen facilitates delivery of the lead over the tether to be received in the channel of the stent-like fixation member.

In Example 19, the system according to any one of Examples 16-18, wherein the stent-like fixation member further includes a tether coupled to the distal end of the stent-like fixation member and extending in a proximal direction away from the stent-like fixation member and the lead body further includes a guide feature coupled to an outer surface of lead body such that the lead body is adapted to track alongside the tether to be received in the channel of the stent-like fixation member.

In Example 20, the system according to any one of Examples 16-19, wherein the stent like fixation member further includes a tether coupled to and extending in a proximal direction away from the stent-like fixation member, the tether comprising one or more conductors extending within the tether from a proximal end to a distal end of the tether and one or more electrodes located on an outer surface of the stent-like fixation member and operatively coupled to the one or more conductors extending within the tether.

In Example 21, the system according to any one of Examples 16-20, wherein the stent-like member further includes an insulative coating or sheath.

Example 22 is a method for securing and stabilizing a medical electrical lead in a patient's internal jugular vein at a location adjacent a region of a vagus nerve to be stimulated located within the patient's carotid sheath, the method comprising: advancing a medical electrical lead into a patient's internal jugular vein to a location adjacent the region of the vagus nerve to be stimulated, the lead comprising a lead body extending from a proximal end to a distal end, a conductor extending within the lead body from the proximal end in a direction towards the distal end, and an electrode region comprising at least one electrode located on the lead body and operatively coupled to the conductor, the electrode adapted to transvasculary deliver an electrical stimulation pulse to the target region of the vagus nerve from a location within the internal jugular vein; advancing an independent stent-like fixation member retained in a collapsed configuration within a delivery catheter to a location adjacent the electrode region of the medical electrical lead; and deploying the independent stent-like fixation member from the delivery catheter such that the stent-like fixation member transitions from the collapsed configuration to an expanded configuration, wherein in the expanded configuration, the stent-like fixation member contacts and engages the electrode region of the lead and urges the electrode region including the at least one electrode into a vessel wall adjacent the region of the vagus nerve to be stimulated.

In Example 23, the method according to Example 22, wherein the lead body further includes a three-dimensional spiral shape defining a lumen and the stent-like fixation member is advanced into the lumen of the three-dimensional spiral shape.

In Example 24, the method according to Examples 22 or 23, further comprising repositioning the electrode region by rotating the stent-like fixation member after the stent is partially expanded.

In Example 25, the method according to any one of Examples 22-24, further comprising partially deploying the stent-like fixation member from the delivery catheter until the stent-like fixation member contacts and engages the electrode region and then, rotating the partially deployed stent-like fixation member to reposition the electrode region within the internal jugular vein.

Example 26 is a method for securing and stabilizing a medical electrical lead in a patient's internal jugular vein at a location adjacent a region of a vagus nerve to be stimulated located within the patient's carotid sheath, the method comprising: advancing a stent-like fixation member retained in a collapsed configuration within a delivery catheter to a location within the internal jugular vein adjacent the region of the vagus nerve to be stimulated, the stent-like fixation member comprising a concave channel sized and shaped to receive the lead therein extending from a proximal end to a distal end of the stent-like member and a tether coupled to the distal end of the stent-like member and extending in a proximal direction away from the stent-like fixation member; deploying the stent-like fixation member from the delivery catheter such that the stent-like fixation member transitions from the collapsed configuration to an expanded configuration and anchors in the vein; and advancing a medical electrical lead including a lumen over the tether such that a portion of the medical electrical lead comprising at least one electrode is received and retained within the concave channel of the stent-like fixation member.

In Example 27, a method according to Example 26, wherein the stent-like fixation member further includes one or more electrodes located on an outer surface of the stent-like fixation member and operatively coupled to one or more conductors extending within the tether and wherein the method further includes the steps of partially deploying the stent-like fixation member and acutely stimulating the vagus nerve using the one or more electrodes to identify an optimal location for delivering electrical stimulus therapy to the vagus nerve.

Example 28 is a method for securing and stabilizing a medical electrical lead in a patient's internal jugular vein at a location adjacent a region of a vagus nerve to be stimulated located within the patient's carotid sheath, the method comprising: advancing a stent-like fixation member retained in a collapsed configuration within a delivery catheter to a location within the internal jugular vein adjacent the region of the vagus nerve to be stimulated, the stent-like fixation member comprising a concave channel sized and shaped to receive the lead therein extending from a proximal end to a distal end of the stent-like member and a tether coupled to the distal end of the stent-like member and extending in a proximal direction away from the stent-like fixation member; deploying the stent-like fixation member from the delivery catheter such that the stent-like fixation member transitions from the collapsed configuration to an expanded configuration; and advancing a medical electrical lead including a guide feature alongside the tether such that a portion of the medical electrical lead comprising at least one electrode is received and retained within the concave channel of the stent-like fixation member.

In Example 29, the method according to Example 28, wherein the stent-like fixation member further includes one or more electrodes located on an outer surface of the stent-like fixation member and operatively coupled to one or more conductors extending within the tether and wherein the method further comprises the steps of partially deploying the stent-like fixation member and acutely stimulating the vagus nerve using the one or more electrodes, rotating the stent to identify an optimal location for delivering electrical stimulus therapy to the vagus nerve and advancing the lead into the channel once the proper orientation is obtained.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a system for stimulating a region of a patient's vagus nerve located within the carotid sheath according to an embodiment of the present invention.

FIG. 2 is a schematic view of an electrode according to an embodiment of the present invention.

FIGS. 3A and 3B are schematic views of a fixation member for use with the system as shown in FIG. 1 according to various embodiments of the present invention.

FIGS. 4A-4C are close-up, schematic views of the system shown in FIG. 1 according to various embodiments of the present invention.

FIG. 5A is a close-up, schematic view of a system according to an embodiment of the present invention prior to implantation of the lead.

FIG. 5B is a close-up, schematic view of the system shown in FIG. 5A according to an embodiment of the present invention after implantation of the lead.

FIG. 6 is a close-up, schematic view of a system for stimulating a region of a patient's vagus nerve located within the carotid sheath according to another embodiment of the present invention.

FIGS. 7A-7B are close-up, schematic view of a system according to an embodiment of the present invention prior to implantation and during delivery of a lead.

FIG. 7C is a close-up, schematic view of the system shown in FIGS. 7A and 7B according to an embodiment of the present invention after implantation of the lead.

FIG. 8A is a close-up, schematic view of a system according to yet another embodiment of the present invention prior to implantation of a lead.

FIG. 8B is a close-up schematic view of the system shown in FIG. 8A according to an embodiment of the present invention after implantation of the lead.

FIG. 9 is a block diagram of a method according to an embodiment of the present invention.

FIG. 10 is a block diagram of a method according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

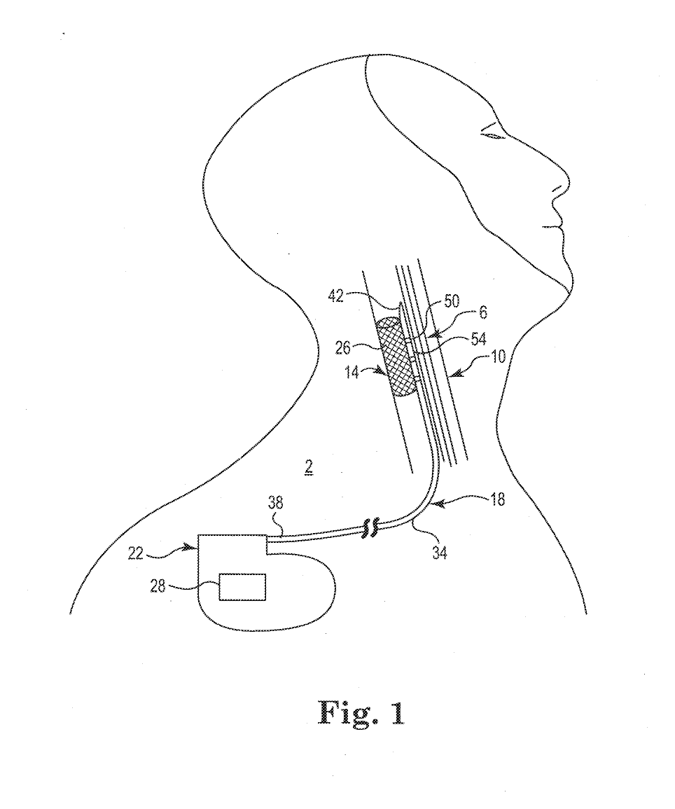

FIG. 1 shows a system 2 for stimulating a region of a patient's vagus nerve 6 located within the carotid sheath 10. The carotid sheath 10 is a tube-shaped fascia wrapping the common carotid artery (not shown), the internal jugular vein (IJV) 14, and the vagus nerve 6. The system 2 includes a neurostimulation lead 18 coupled to an implantable medical device (IMD) 22 such as, for example, a pulse generator and an independent fixation member 26 that is provided separate from the lead 18. According to various embodiments, the independent fixation member 26 is delivered to the implant site independently and separate from the lead 18. The lead 18 is secured within the internal jugular vein 14 at a target location adjacent a region of the vagus nerve 6 to be stimulated using the fixation member 26.

The neurostimulation lead 18 includes an elongated, insulative lead body 34 and extending from a proximal end 38 to a distal end 42. The lead 18 is coupled to the IMD 22 via a connector (not shown) located at the proximal end 38 of its lead body 34. The lead body 34 is flexible and, in some embodiments, may have a circular cross-section. Alternatively, in other embodiments the lead body 34 (or portions thereof) may have non-circular (e.g., elliptical) cross-sectional shapes. In some embodiments, the lead body 34 can include multiple lumens. For example, in one embodiment, the lead body 34 can include one or more guide lumens to receive a guide member such as a guidewire or stylet or to facilitate over-the-wire delivery of the lead 18 to the desired implantation site.

According to various embodiments, the neurostimulation lead 18 can include a plurality of conductors including individual wires, coils, or cables extending within the lead body 34 from the proximal end 38 in a direction towards the distal end 42 of the lead body 34. The conductors can be insulated with an insulator such as silicone, polyurethane, ethylene tetrafluoroethylene, or another biocompatible, insulative polymer. In one exemplary embodiment, the conductors have a co-radial design. In this embodiment, each individual conductor is separately insulated and then wound together in parallel to form a single coil. In another exemplary embodiment, the conductors have a co-axial, non-co-radial configuration. In still other embodiments, one or more of the conductors is a stranded cable conductor each routed through one of the aforementioned lumens in the lead body 34. In short, any conductor configuration can be employed in the lead according to the various embodiments of the present invention.

The lead 18 includes at least one electrode 50 located on an electrode region 54 of the lead body 34. In one embodiment, the lead 18 is a bipolar lead 18 including a pair of electrodes 50 located on an electrode region 54 of the lead body. In another embodiment, the lead 18 is a quadripolar lead 18 having four electrodes 50 located on an electrode region 54 of the lead body. In still other embodiments the lead 18 can have an electrode array including more than four electrodes 50. Each conductor extending within the lead body 34 is adapted to be connected to an individual electrode 50 located on an electrode region 54 of the lead body 34 in a one-to-one manner allowing each electrode 50 to be individually addressable. Additionally, each electrode 50 located on the lead body 34 can be programmed by the IMD 22 to assume a positive (+) or negative (−) polarity to create a particular stimulation field when current, for example, is applied thereto.

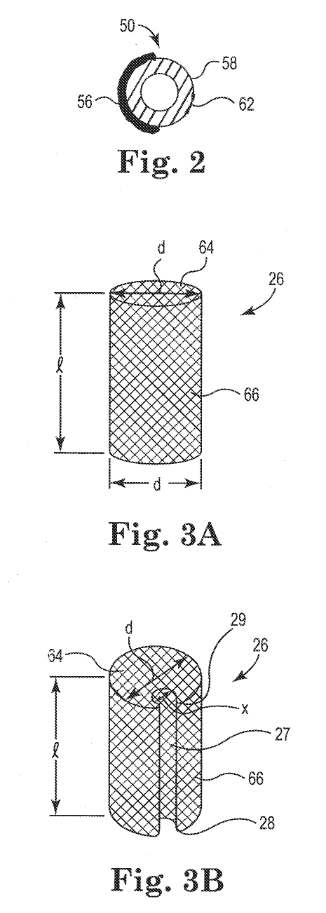

According to various embodiments, the electrode 50 is adapted to transvascularly deliver an electrical stimulation pulse across the wall of the IJV 14 to the vagus nerve 6. The electrode 50 can be a ring or partial ring electrode. In some embodiments, as shown in FIG. 2, the electrode 50 may include a masked portion 56 and an unmasked portion 58 having an electrically active surface 62. Upon implantation of the lead 18 in the IJV 14, the electrode region 54 of the lead 18 including the at least one electrode 50 is oriented such that the electrically active surface 62 of the electrode 50 is oriented in a direction towards the region of the vagus nerve 6 to be stimulated and the masked portion 56 of the electrode 50 is oriented away from the vagus nerve 6 so as to shield other areas of the anatomy from undesired or unintended stimulation.

FIGS. 3A and 3B are schematic views of the fixation member 26 according to various embodiments of the present invention. As shown in FIGS. 3A and 3B, the fixation member 26 is an elongated stent-like fixation member 26 that is provided separate from the lead 18. In some embodiments, as shown in FIG. 3A, the elongated stent-like fixation member 26 is generally cylindrical. In other embodiments, as shown in FIG. 3B, the stent-like fixation member 26 includes a pre-formed concave channel 27 sized and shaped to receive a portion of the lead body 34 extending from a proximal end 28 to a distal end 29 of the stent-like fixation member 26. The channel 27 is sized and shaped to receive a portion of a lead body such as, for example, lead body 34. In one embodiment, the channel 27 has a generally C-shaped cross-section. In another embodiment, the channel 27 has a generally U-shaped cross-section. Additionally, in some embodiments, the channel 27 has an inner diameter x generally corresponding to or slightly larger than an outer diameter of the portion of the lead body to be received therein. In other embodiments, the stent-like structure defining the channel 27 is sufficiently resilient such that the channel 27 is adapted to expand such that the inner diameter x increases so as to accommodate a lead body having a larger diameter.

The stent-like fixation member 26, according to the embodiments described above, can be constructed from a variety of biocompatible materials. In some embodiments, the stent-like fixation member 26 is constructed from a wire mesh such as, for example a stainless steel or Nitinol wire mesh. In other embodiments, the stent-like fixation member 26 can be constructed from an insulative, biocompatible polymer. In still another embodiment, the stent-like fixation member 26 can be constructed from a bio-resorbable polymer such that the stent-like fixation member 26 dissolves over time. Additionally, in some embodiments, the stent-like fixation member 26 can include an insulative coating or sheath provided over an outer surface of the stent-like fixation member 26 to prevent or avoid electrical contact with the lead's electrodes.

The elongated stent-like fixation member 26 is adapted to transition from a collapsed configuration for delivery to the implant site within the IJV 14 to an expanded configuration, as shown in FIG. 1. In one embodiment, the stent-like fixation member 26 is balloon-expandable. In another embodiment, the stent-like fixation member 26 is self-expanding. According to various embodiments, when in the expanded configuration, the stent-like fixation member 26 expands to an outer diameter dthat is greater than an inner diameter of the IJV 14 such that it places a sufficient amount of a radial expansion force on the vessel walls of the IJV 14 and on the electrode region 54 of the lead 18 so as to secure and stabilize the electrode region 54 of the lead 18 in the IJV 14. While the stent-like fixation member 26 is illustrated as a cylinder, in some embodiments the stent-like fixation member 26 is sufficiently resilient such that it deforms or wraps around and engages an outer surface of the lead body 34 forming a channel around the lead body 34 and urging the lead body 34 into the vessel walls of the IJV 14. In another embodiment, as shown in FIG. 3B, the stent-like fixation member 26 includes a pre-formed concave channel 27 sized and shaped to engage an outer surface of the lead body 34. Additionally, according to some further embodiments of the present invention, the stent-like fixation member 26 has a length l that is greater than an overall length of the electrode region 54.

In some embodiments, the elongated stent-like member 26 can include a coating on its inner surface 64 and/or outer surface 66. For example, the elongated stent-like member 26 can include a non-thrombogenic coating on its inner surface 64 and/or outer surface 66. In still other embodiments, the elongated stent-like member 26 can include a drug eluting coating on its inner and/or outer surfaces 64 and 66.

In another embodiment of the present invention, the elongated stent-like member 26 can include a polymer sheath lining the inner surface 64 and/or outer surface 66 of the stent-like member 26. The polymer sheath can be fabricated from a material that is adapted to prevent tissue ingrowth through the stent structures. Additionally, the material from which the polymer sheath is fabricated is adapted to expand with the stent like fixation member 26 as the stent-like fixation member 26 is deployed.

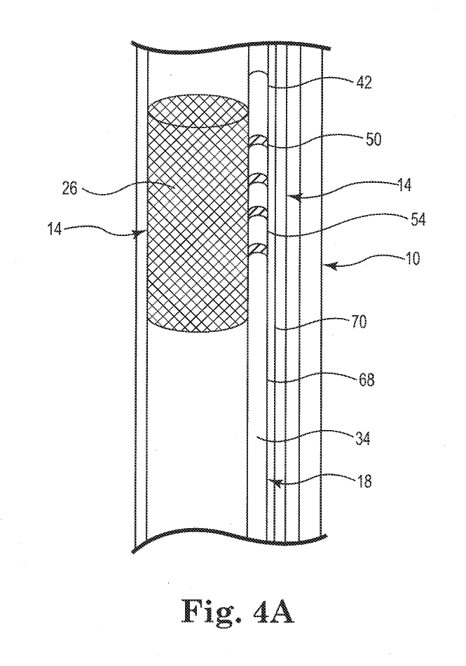

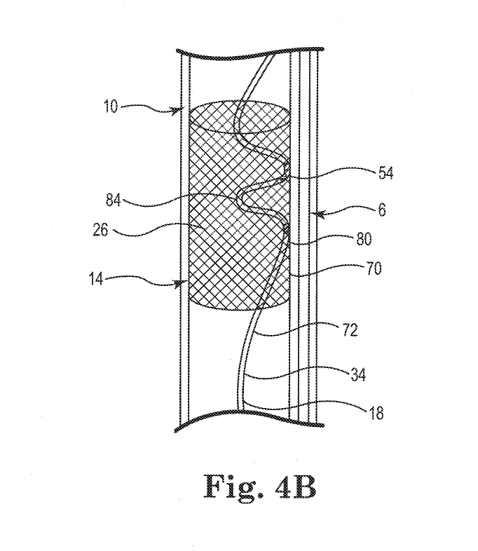

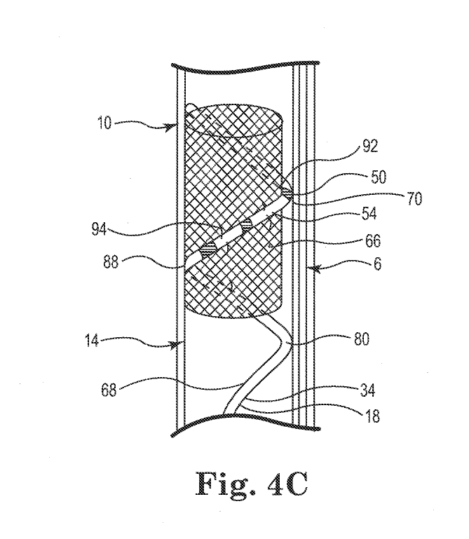

FIGS. 4A-4C are close up schematic views of the electrode region 54 of a lead 18 according to various embodiments secured within the IJV 14 adjacent a target region of the vagus nerve 6 using a stent-like fixation member 26, such as described in detail above according to the various embodiments. The electrode region 54 can be a portion of the lead body 34 located between the proximal end 38 and the distal end 42 of the lead body 34. In one embodiment, the electrode region 54 is located at a distal region 68 of the lead body 34, as shown in FIGS. 4A-4C. In another embodiment, the electrode region 54 can be located on a middle region of the lead body 34 (not shown). In some embodiments, the lead 18 can have more than one electrode region 54.

In one embodiment, as shown in FIG. 4A, the electrode region 54 of the lead 18 is a generally straight portion of the lead body 34 including one or more electrodes 50. The electrode region 54 of the lead 18 can be delivered to the implant site within the IJV 14 using a variety of techniques know to those of skill in the art. The electrode region 54 is oriented within the IJV such that the electrically active region (see for example, FIG. 2) of the electrode 50 is oriented in a direction towards the vagus nerve 6. The stent-like fixation member 26 is then delivered to a location adjacent the electrode region 54 and expanded such that it contacts and engages the electrode region 54, urging the electrically active region of at least one of the electrodes 50 located on the distal portion into contact with the vessel wall 70 adjacent the vagus nerve 6. In some embodiments, partial masking of the electrode(s) 50 or an insulative coating or sheath provided over the outer surface 66 of the stent-like member 26 can be used to insulate the stent-like member 26 from the electrical contact with the electrode(s) 50. In another embodiment, the stent-like member 26 can be a non-conductive, polymeric stent-like fixation member.

In other embodiments, as shown in FIGS. 4B and 4C, the electrode region 54 of the lead 18 is a shaped region 80 of the lead body 34 including one or more electrodes 50. The shaped region 80 of the lead body 34 can include a variety of pre-formed shapes including two dimensional shapes such as, for example, the two-dimensional sine wave 84 shown in FIG. 4B or three-dimensional shapes such as the spiral shape 88 shown in FIG. 4C. The shaped region 80 of the lead body 34, in addition to the stent-like fixation member 26, can be used to further secure and stabilize the electrode region 54 of the lead 18 in the IJV 14 such that the electrically active surface of at least one electrode 50 located on the electrode region 54 is oriented in a direction towards the region of the vagus nerve 6 to be stimulated.

As shown in FIGS. 4A-4C, the stent-like fixation member 26 is independent and separate from the lead 18. According to some embodiments, as shown in FIGS. 4A and 4C, the stent-like fixation member 26 is delivered separately to a location alongside the electrode region 54 of the lead 18 and then expanded against the electrode region 54, urging the electrode region 54 into the vessel wall 70 adjacent the vagus nerve 6 to secure and stabilize the electrode region 54 of the lead 18 within the IJV 14. In other embodiments, as shown in FIG. 4C, the stent-like fixation member 26 can be inserted within the shaped region 80 of the lead body 34 and then expanded to secure and stabilize the shaped region 80 of the lead body 34 within the IJV 14. More particularly, the stent-like fixation member 26 is inserted into a lumen 92 formed by the spiral 88 and then expanded such that the spiral 88 wraps around an outer surface 66 of the stent-like fixation member 26, forcing at least a portion of the spiral 88 including an electrode 50 located thereon into the vessel wall 70 adjacent the vagus nerve 6. In some embodiments, partial masking of the electrode(s) 50 or an insulative coating or sheath provided over the outer surface 66 of the stent-like fixation member 26 can be used to insulate the stent-like fixation member 26 the electrical contact with the electrode(s) 50. In another embodiment, the stent-like fixation member 26 itself can be fabricated from an insulative material, polymeric material such that it does not make electrical contact with the electrode(s).

In some embodiments, the stent-like fixation member 26 can be used to further orient the electrode region 54 of the lead body 34 within the IJV 14. The stent-like fixation member 26 can be at least partially deployed from a delivery catheter until it frictionally engages at least a portion of the electrode region 54 of the lead body 34. Once frictionally engaged with the electrode region 54, the partially expanded fixation member 26 can be rotated, thereby causing rotation of the electrode region 54 of the lead body 34 within the IJV 14. In one further embodiment, the stent-like fixation member 26 may be provided with a tacky or adhesive surface coating over a least a portion of its outer surface 66 to enhance frictional engagement of the stent-like fixation member 26 with the electrode region 54 of the lead body 34. In another further embodiment, the stent-like fixation member 26 can include one or more prongs or hooks configured to engage the lead body 34 in a cooperative manner. In still yet another embodiment, as shown in FIG. 4C, the lead body 34 can include one or more projections 94 such as prongs, hooks or tines that are adapted to engage the mesh outer surface 66 of the stent-like fixation member 26. Once the stent-like fixation member 26 is engaged with the lead body 34, the stent-like fixation member 26 can be rotated, thereby causing rotation of the lead body 34.

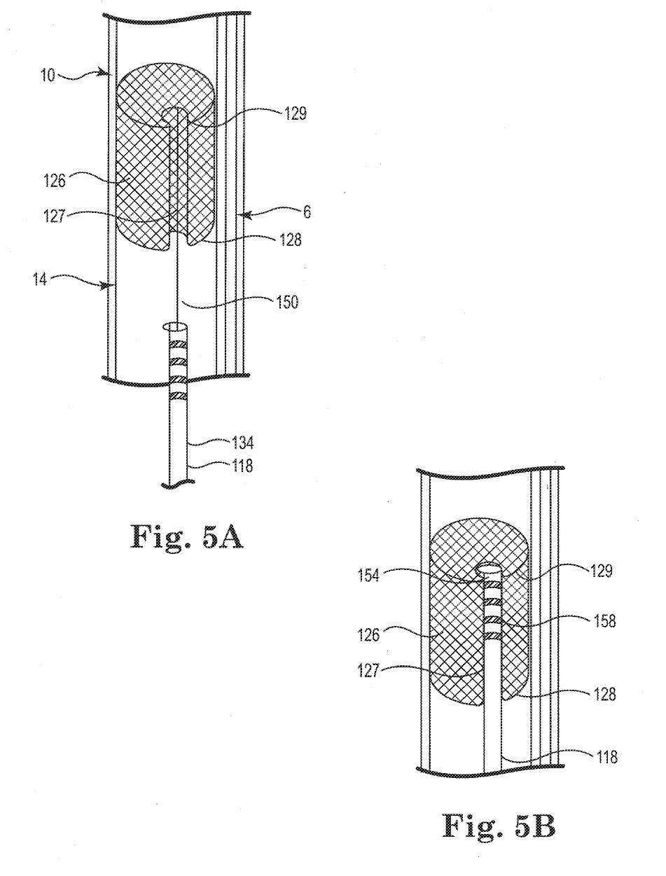

FIG. 5A is a close-up schematic view of a stent-like fixation member 126 deployed in a patient's IJV 14 prior to the delivery and implantation of a lead 118. FIG. 5B is a close-up, schematic view of stent-like fixation member 126 and lead 118 after delivery and implantation of the lead 118. As shown in FIG. 5A, the stent-like fixation member 126 includes many of those same features as the fixation members 26 described above in reference to FIGS. 3A and 3B and also includes a channel 127. The channel 127 extends from a proximal end 128 to a distal end 129 of the stent-like fixation member 126. According to various embodiments, the channel 127 is sized and shaped to engage and receive the lead body 134 therein. In one embodiment, the channel 127 has a generally C-shaped cross-section. In another embodiment, the channel 127 has a generally U-shaped cross-section. Additionally, the channel 127 has an inner diameter that generally corresponds to and/or is slightly larger than the outer diameter of the lead body 134.

According to some embodiments, as best shown in FIG. 5A, the stent-like fixation member 126 also includes a tether 150 coupled to the distal end 129 of stent-like fixation member 126 and extending within the channel 127. The tether 150 facilitates delivery of the lead body 134 within the channel 127. The tether 150 can be made from a variety of materials. For example, in one embodiment, the tether 150 can be fabricated from a polymer or a material similar to those used to fabricate sutures. In other embodiments, the tether can be fabricated from a bio-resorbable material that is capable of being dissolved over time in the body environment. In still yet another embodiment, the tether 150 can include one or conductors extending within the tether 150 from a proximal end to a distal end surrounded by one or more layers of an insulation.

In some embodiments, the lead body 134 includes a lumen (not shown) extending within the lead body from its proximal end to its distal end to facilitate delivery of the lead 118 over the tether 150 to be received within the channel 127 as shown in FIG. 5B. In one embodiment, the lead body 134 is delivered over the tether 150 and received within the channel 127 such that a distal end 154 of the lead body 134 is adjacent to the distal end 129 of the stent-like fixation member 126. The channel 127 and tether 150 of the stent-like fixation member 126 cooperates with the lead body 134 to secure and stabilize the lead body 134 at a target location within the IJV 14 adjacent the vagus nerve 6. More particularly, the channel 127 and tether 150 cooperate to secure and stabilize the lead body 134 at the target location in the IJV such that at least one electrode 158 is oriented in a direction towards the vagus nerve 6.

In another embodiment, the lead body 134 can include a loop feature or other guide feature that facilitates advancement of the lead body 134 alongside the tether 150 using the tether 150 as a guide or track. In this embodiment, the lead body 134 can be advanced alongside the tether 150 using a stylet or other delivery tool to deliver the lead body 134 to the desired location.

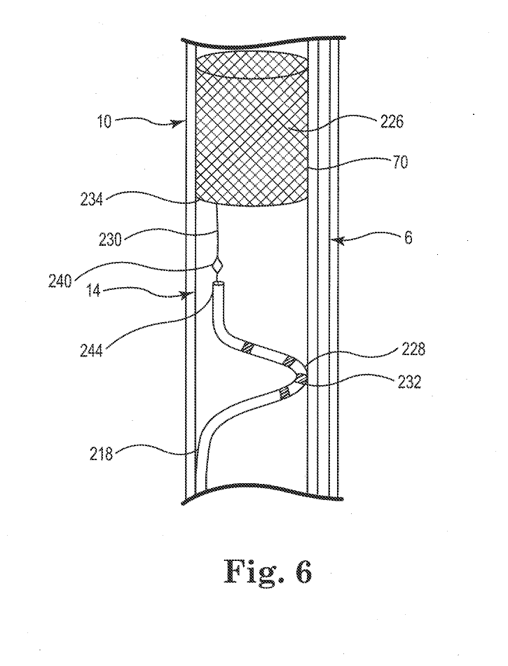

FIG. 6 is a close-up schematic view of a system 200 including stent-like member 226 deployed within a patient's IJV 14 and used to secure and stabilize a lead 218 at a location adjacent a region of the vagus nerve 6 to be stimulated according to yet another embodiment of the present invention. As shown in FIG. 6, the stent-like member 226 is similar to stent-like members 26 and 126 described above according to the various embodiments of the present invention and includes many or all of those same features. The stent-like member 226 is expanded such that it contacts the vessel walls 70 of the IJV 14 such that it is secured and stabilized within the IJV 14 adjacent the vagus nerve 6. According to one embodiment, as shown in FIG. 6, the stent-like member 226 includes a tether 230 coupled to and extending in a proximal direction away from its proximal end 234.

The tether 230 provides a track or guide for delivery of the lead 218 to the implant location within the IJV 14 adjacent the vagus nerve 6. In one embodiment, the lead 218 can include at least one lumen (not shown) that facilitates its delivery over the tether 230. In another embodiment, the lead 218 can include a loop feature or other guide feature that facilitates delivery of the lead 218 alongside the tether 230 using the tether 230 as a guide or track. The lead 218 can include a pre-formed shape 228 having a two-dimensional or three dimensional shape. As shown in FIG. 6, the pre-formed shape 228 can be a three dimensional spiral shape. According to various embodiments, one or more electrodes 232 can be located on the shaped region 228.

In one embodiment, the shaped region 228 is adapted to transition from a collapsed configuration suitable for delivery to an expanded configuration. Tension can be placed on the tether 230 to retain the lead in a collapsed configuration during delivery of the lead 218 over the tether 230. The lead 218 transitions from its collapsed configuration to its expanded configuration where it returns to its pre-formed shape by relaxing the tension on the tether 230. In the expanded configuration, the shaped region 228 places a sufficient lateral force on the vessel walls 70 so as to secure and stabilize the lead 218 at the implant location within the IJV, urging at least one electrode 232 into the vessel walls 70.

In one embodiment, as shown in FIG. 6, the tether 230 also includes a stop 240. The stop 240 is located at a fixed distance relative to the proximal end 234 of the stent-like fixation member 226. During deployment of the system 200, the lead 218 is advanced over the tether 230 until it reaches the stop 240. The lead 218 is located proximal to the stent-like fixation member 226 such that the distance between the distal end 244 of the lead 218 and the proximal end 234 of the stent-like member 226 is fixed. In one embodiment, to further secure and stabilize the lead 218 at the implantation location within the IJV 14, the lead 128 can be secured at the distal stop 240 and the proximal end of the lead 218 by the application of the a cap or by tying a knot in the tether 230 as the tether 230 exits the proximal end of the lead 218. As a result, movement of the lead 218 over the tether 230 is prevented once the lead 218 has been implanted.

In another embodiment, the proximal end 234 of the stent-like fixation member 226 can serve as the stop 240. In this example, the lead 218 is advanced along or over the tether 230 until the distal end 244 of the lead 218 reaches the proximal end 234 of the stent-like fixation member 226. The lead 218 can then be secured and stabilized at the implantation location within the IJV by capping or tying a knot in the tether as it exits the proximal end of the lead 218 as discussed above.

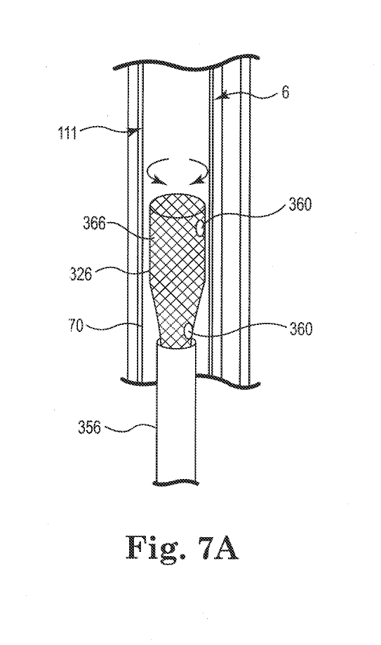

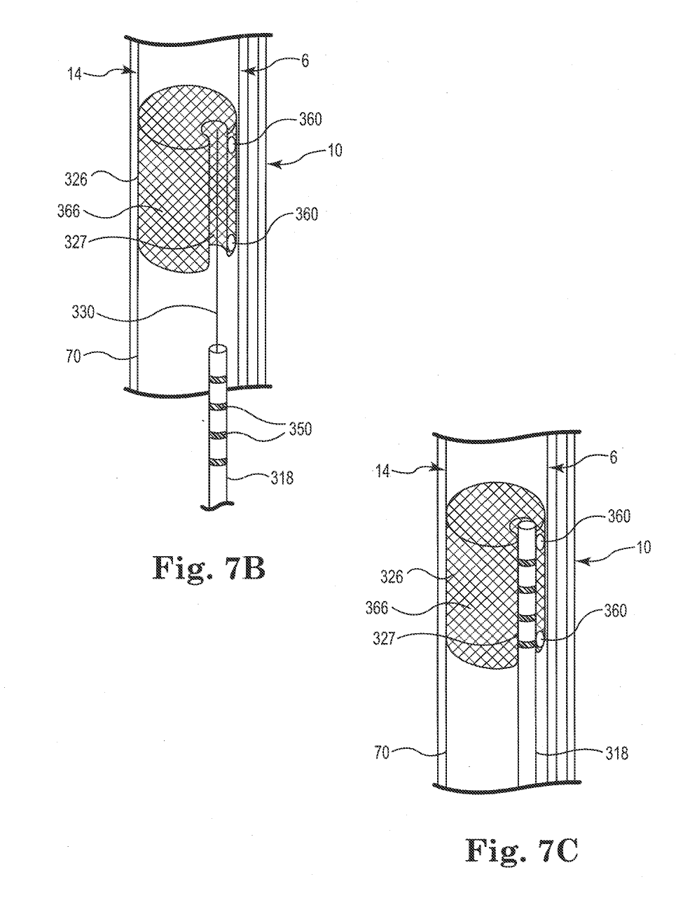

In other embodiments of the present invention as shown in FIGS. 7A-7C, a stent-like fixation member 326 can include one or more electrodes 360 located on an outer surface 366 of the stent-like fixation member 326 and operatively coupled to one or more conductors extending within the tether 330 from the proximal end to the distal end of the tether 330. The stent-like fixation member 326 includes many or all of the same features as the stent-like fixation members 26, 126 and 226 described above according to the various embodiments. During delivery, the stent-like fixation member 326 can be partially deployed from a delivery catheter 356 so that the electrodes 360 can be positioned closer to the vessel walls 70 of the IJV 14. As shown in FIG. 7A, the partially deployed stent-like fixation member 326 can be rotated and repositioned until an optimal location for delivery of stimulation to the vagus nerve 6 has been indentified using the electrodes 360 located on an outer surface 366 of the stent-like fixation member 326. In some embodiments, the electrode(s) 360 can be used to acutely stimulate the vagus nerve 6 until a desired threshold for physiological response used to evaluate the efficacy of the stimulation has been achieved. In other embodiments, the electrode(s) 260 can be used as sensing electrodes (sensing nerve traffic signal, artery pulsation via impedance change, etc.) in order to locate the nerve for final lead positioning. Once the region of the nerve 6 to be stimulated has been identified, the stent-like member 326 can then be fully expanded at the implantation location as shown in FIG. 7B. After expansion of the stent-like member 326, the lead body 318 can then be delivered over or alongside the tether 330 and received within the channel 327 such that electrode(s) 350 located on the lead 318 are positioned within the internal jugular vein adjacent the vagus nerve 6 as shown in FIG. 7C. Electrical stimulus therapy can then be delivered to the vagus nerve 6 using the electrodes 350 located on the lead 318.

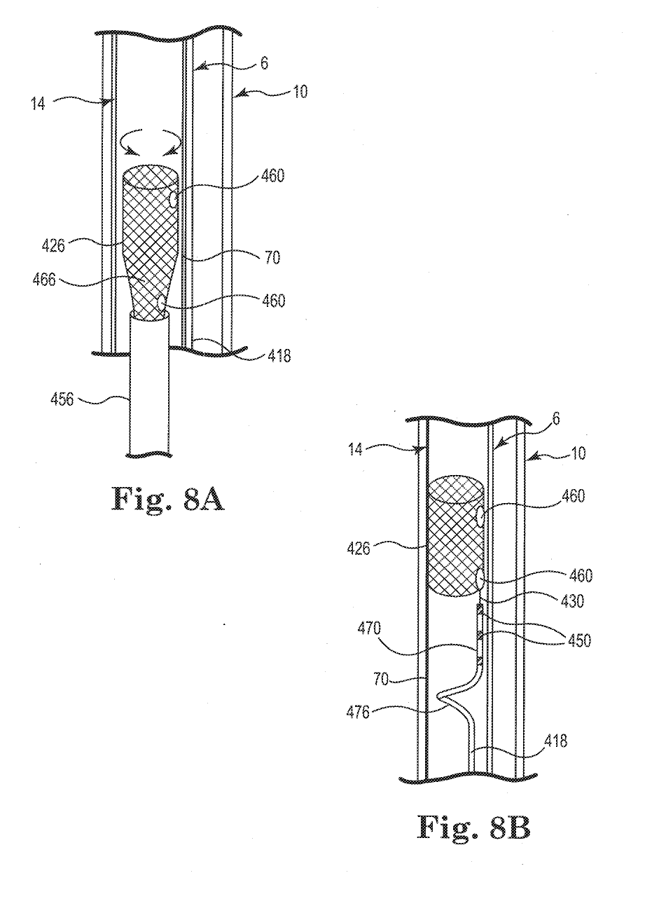

FIGS. 8A and 8B are close-up schematic views of a stent-like fixation member 426 according to yet another embodiment of the present invention. The stent-like fixation member 426 can include many or all of the same features as the stent-like fixation members 26, 126, 226 and 326 described above according to the various embodiments. In addition, as shown in FIGS. 8A and 8B, the stent-like fixation member 426 includes one or more electrodes 460 located on an outer surface 466 of the stent-like fixation member 426 and operatively coupled to one or more conductors extending within the tether 430 coupled to the stent-like fixation member 426.

During delivery, as shown in FIG. 8A, the stent-like fixation member 426 can be partially deployed from a delivery catheter 456 so that the electrodes 460 can be positioned closer to the vessel walls 70 of the IJV 14. As indicated in FIG. 8A, the partially deployed stent-like fixation member 426 can be rotated and repositioned until an optimal location for delivery of stimulation to the vagus nerve 6 has been identified using the electrodes 460 located on an outer surface 466 of the stent-like fixation member 426. The stent-like member 426 can then be fully expanded at the implantation location as shown in FIG. 8B. After expansion of the stent-like member 426, the lead 418 can then be delivered over or alongside the tether 430. The lead 418 would track the tether 430 and stop near the proximal electrode 460 to position the electrodes 450 located on the lead 418 at the desired location adjacent the vagus nerve 6. In one embodiment, the electrodes 450 are located on a straight portion 470 of the lead 418 such that they are adapted to be placed in parallel alignment with the adjacent region of the vagus nerve 6. In a further embodiment, the lead 418 can include a pre-formed region such as a pre-formed spiral region 476 as shown in FIG. 8B which can be used to further secure and stabilize the electrode region of the lead 418 at the desired position adjacent the vagus nerve 6. As described above, the tether 430 can be capped or knotted at the proximal end where the tether 430 exits the proximal end of the lead as a further mechanism for securing and stabilizing the lead 418 at the desired position.

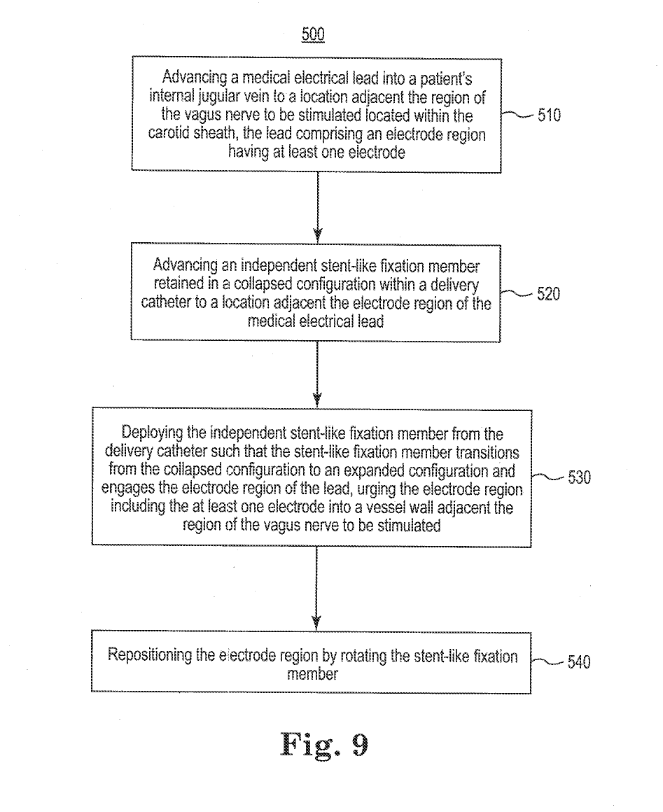

FIG. 9 outlines a method 500 of securing and stabilizing a medical electrical lead, such as described above according to the various embodiments, at a location within the IJV 14 for stimulating a region of the vagus nerve 6. According to one embodiment of the method 500, the lead is advanced within a patient's IJV 14 to a location adjacent the region of the vagus nerve 6 to be stimulated (Block 510). The lead can be delivered to the target location using a variety of lead delivery techniques. In one embodiment, the lead is advanced over a guidewire to the location within the IJV 14. In other embodiments, the lead can be delivered using a guide catheter or a stylet. Acute stimulation of the vagus nerve 6 can be used to determine whether placement of the lead at that location results in optimal stimulation of the vagus nerve 6 to produce a desired physiological effect. Physiological effects that could be produced and subsequently monitored include laryngeal muscle vibration from activation of the RLN (branch of the vagus nerve), heart rate, blood pressure, voice alteration or electroencephalogram signals. The lead can be repositioned as necessary until an optimal position for stimulation resulting in a desired physiological effect is identified.

Next, according to one embodiment, a delivery catheter can be used to advance a stent-like member to the location adjacent the electrode region on the lead (Block 520). The stent-like member is retained in a collapsed configuration during delivery by the delivery catheter. The stent-like member is then deployed from the delivery catheter and the stent-like member is transitioned from its collapsed configuration to an expanded configuration (Block 530). In one embodiment, the stent-like member is transitioned by the expansion of a balloon inserted within the stent-like member. In another embodiment, the stent-like member is configured to self-expand upon deployment from the delivery catheter. In the expanded configuration, the stent-like member engages an electrode region of the lead, urging the electrode region including at least one electrode into a vessel wall adjacent the region of the vagus nerve 6 to be stimulated (Block 530). In a further embodiment, once deployed, the stent-like member can be used to reposition the electrode region of the lead by rotating the stent-like fixation member in clock-wise or counter clockwise direction (Block 540).

In yet another embodiment of the method, the stent-like fixation member can be partially deployed from the delivery catheter until it contacts and engages an electrode region of the lead. The stent-like member can then be used to reposition the electrode region by rotating the electrode region in a clockwise or counter clockwise manner until an optimal location for stimulation of the vagus nerve is achieved. The stent-like member can then be fully deployed from the delivery catheter to secure and stabilize the electrode region of the lead at that location.

In still yet another embodiment of the method, the lead can include a pre-formed, shaped region. The pre-formed, shaped region can have a two-dimensional or three-dimensional shape. The pre-formed, shaped region provides an additional mechanism for securing and stabilizing the lead within the IJV 14. The lead can be advanced to a target location within the IJV 14 adjacent the vagus nerve 6 using a stylet or guidewire. Upon reaching the target location, the guidewire or stylet is removed from the lead such that the shaped region of the lead returns to its pre-formed shape and is secured within the IJV 14. The stent-like member is then advanced to a location adjacent to or within the pre-formed shape and then expanded urging the pre-shaped region of the lead against the vessel walls of the IJV 14, further securing and stabilizing the lead within the IJV 14. For example, in one embodiment, the pre-formed shaped region of the lead is a spiral and the stent-like member is advanced to a position within the lumen defined by the spiral and then transitioned from its collapsed configuration to its expanded configuration.

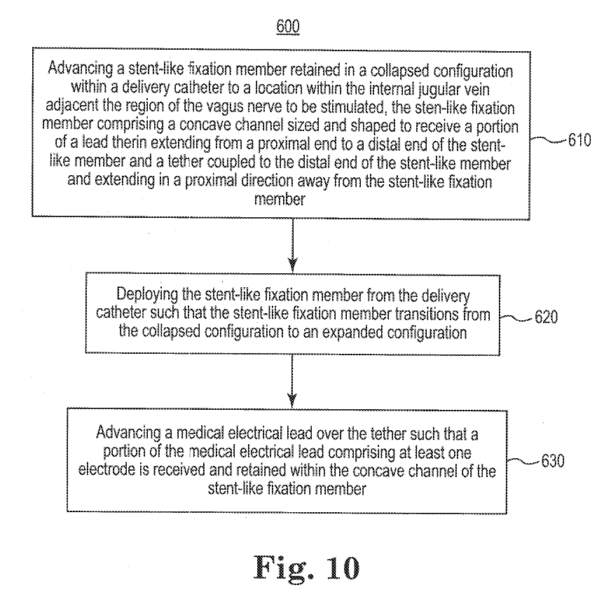

FIG. 10 outlines a method 600 of securing and stabilizing a medical electrical lead according to another embodiment of the present invention. The method includes advancing a stent-like fixation member retained in a collapsed configuration within a delivery catheter to a location within the internal jugular vein and deploying the stent-like member from the catheter such that the stent-like member transitions from the collapsed configuration to an expanded configuration (Blocks 610 and 620). The stent-like fixation member includes a concave channel sized and shaped to receive a portion of a lead therein extending from a proximal end to a distal end of the stent-like member. The stent-like member also includes a tether coupled to a distal end of the stent-like member and extending in a proximal direction away from the stent-like member (Block 610).

After the stent-like member is expanded to secure and stabilize it within the IJV 14, a lead is then advanced over or alongside the tether until it is received within the channel of the stent-like member (Block 630). In one embodiment, the portion of the lead that is advanced and received within the channel includes at least one electrode.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A lead system for stimulating a region of a vagus nerve from a location within an internal jugular vein, the system comprising:
a medical electrical lead comprising a lead body extending from a proximal end to a distal end, a conductor extending within the lead body from the proximal end in a direction towards the distal end, and an electrode region having a first length along the lead body comprising at least one electrode located on the lead body and operatively coupled to the conductor, the electrode adapted to transvasculary deliver an electrical stimulation pulse to the target region of the vagus nerve from a location within the internal jugular vein; and
a stent-like fixation member independently deployable of the lead, the stent-like fixation member having a second length extending in a direction along a longitudinal axis of the stent-like fixation member that is greater than the first length of the electrode region of the lead, the stent-like fixation member adapted to transition from a collapsed configuration for delivery to a location within the internal jugular vein to an expanded configuration, wherein in the expanded configuration, the fixation member is adapted to contact and engage the electrode region of the medical electrical lead such that the fixation member urges the electrode region including the electrode into a vessel wall adjacent the target region of the vagus nerve.

2. The system according to claim 1, wherein the elongated, stent-like fixation member comprises a concave channel extending from a first end to a second end of the fixation member,

3. The system according to claim 2, wherein the channel has an inner diameter corresponding to the outer diameter of the lead body received therein.

4. The system according to claim 2, wherein the channel is adapted to expand to an increased inner diameter so as to receive and engage the lead body therein.

5. The system according to claim 2, wherein the stent-like fixation member further comprises a tether coupled to the distal end of the stent-like fixation member and extending away from the stent-like member in a proximal direction, wherein the lead body lumen facilitates delivery of the lead over the tether to be received in the channel of the stent-like fixation member.

6. The system according to claim 2, wherein the stent-like fixation member further comprises a tether coupled to the distal end of the stent-like fixation member and extending in a proximal direction away from the stent-like fixation member and the lead body further comprises a guide feature coupled to an outer surface of lead body such that the lead body is adapted to track alongside the tether to be received in the channel of the stent-like fixation member.

7. The system according to claim 1, wherein the stent-like fixation member is sufficiently resilient such that it wraps around and engages an outer surface of the lead body forming a channel around the lead body.

8. The system according to claim 1, wherein the stent like fixation member further comprises a tether coupled to and extending in a proximal direction away from the stent-like fixation member, the tether comprising one or more conductors extending within the tether from a proximal end to a distal end of the tether and one or more electrodes located on an outer surface of the stent-like fixation member and operatively coupled to the one or more conductors extending within the tether.

9. The system according to claim 1, wherein the stent-like fixation member comprises an insulative polymer coating or sheath.

10. The system according to claim 1, wherein the electrode region comprises a pre-formed spiral region defining a lumen.

11. The system according to claim 1, wherein the stent-like member further comprises a tether coupled to and extending away from a proximal end of the stent-like member and wherein the lead body further comprises a lumen extending from the proximal end to the distal end of the lead body, wherein the lead body lumen facilitates delivery of the lead over the tether.

12. The system according to claim 1, wherein the lead body further comprises one or more projections adapted to engage an outer surface of the stent-like fixation member.

13. A method for securing and stabilizing a medical electrical lead in a patient's internal jugular vein at a location adjacent a region of a vagus nerve to be stimulated located within the patient's carotid sheath, the method comprising:
advancing a medical electrical lead into a patient's internal jugular vein to a location adjacent the region of the vagus nerve to be stimulated, the lead comprising a lead body extending from a proximal end to a distal end, a conductor extending within the lead body from the proximal end in a direction towards the distal end, and an electrode region comprising at least one electrode located on the lead body and operatively coupled to the conductor, the electrode adapted to transvasculary deliver an electrical stimulation pulse to the target region of the vagus nerve from a location within the internal jugular vein;
advancing an independent stent-like fixation member retained in a collapsed configuration within a delivery catheter to a location adjacent the electrode region of the medical electrical lead; and
deploying the independent stent-like fixation member from the delivery catheter such that the stent-like fixation member transitions from the collapsed configuration to an expanded configuration, wherein in the expanded configuration, the stent-like fixation member contacts and engages the electrode region of the lead and urges the electrode region including the at least one electrode into a vessel wall adjacent the region of the vagus nerve to be stimulated.

14. The method according to claim 13, further comprising repositioning the electrode region by rotating the stent-like fixation member.

15. The method according to claim 13, further comprising partially deploying the stent-like fixation member from the delivery catheter until the stent-like fixation member contacts and engages the electrode region and then, rotating the partially deployed stent-like fixation member to reposition the electrode region within the internal jugular vein.

16. A method for securing and stabilizing a medical electrical lead in a patient's internal jugular vein at a location adjacent a region of a vagus nerve to be stimulated located within the patient's carotid sheath, the method comprising:
advancing a stent-like fixation member retained in a collapsed configuration within a delivery catheter to a location within the internal jugular vein adjacent the region of the vagus nerve to be stimulated, the stent-like fixation member comprising a concave channel sized and shaped to receive the lead therein extending from a proximal end to a distal end of the stent-like member and a tether coupled to the distal end of the stent-like member and extending in a proximal direction away from the stent-like fixation member;
deploying the stent-like fixation member from the delivery catheter such that the stent-like fixation member transitions from the collapsed configuration to an expanded configuration; and
guiding a medical electrical lead including along the tether such that a portion of the medical electrical lead comprising at least one electrode is received and retained within the concave channel of the stent-like fixation member.

17. The method according to claim 16, wherein the stent-like fixation member further comprises one or more electrodes located on an outer surface of the stent-like fixation member and operatively coupled to one or more conductors extending within the tether and wherein the method further comprises the steps of partially deploying the stent-like fixation member and acutely stimulating the vagus nerve using the one or more electrodes to identify an optimal location for delivering electrical stimulus therapy to the vagus nerve.

18. The method according to claim 16, wherein the lead includes a lumen and is guided over the tether to be received and retained within the concave channel of the stent-like member.

19. The method according to claim 16, wherein the lead includes a guide feature coupled to an outer surface of the lead and the lead is guided alongside the tether to be received and retained within the concave channel of the stent-like member.
